(19) United States
(12) Patent Application Publication
(10) Pub. No.: US 2015/0315634 A1
(43) Pub. Date: Nov. 5, 2015

(54) Calibration of High Resolution Melting

(71) Applicant: Roche Molecular Systems, Inc., Pleasanton, CA (US)

(72) Inventors: Astrid Reiser, Penzberg (DE); Gregor Sagner, Penzberg (DE)

(21) Appl. No.: 14/792,224

(22) Filed: Jul. 6, 2015

Related U.S. Application Data
(63) Continuation of application No. PCT/EP2014/050243, filed on Jan. 8, 2014.

(30) Foreign Application Priority Data
Jan. 10, 2013 (EP) 13150790.7

(51) Int. Cl. C12Q 1/68

(52) U.S. Cl. CPC C12Q 1/6846; C12Q 1/686

(57) ABSTRACT

The present invention refers to a method and a kit for performing temperature calibration in high resolution melting PCR experiments. The present invention further refers to a method for optimal calibration allowing read-out of identical or similar melting temperatures for target and calibrator. The present invention further refers to an apparatus for performing the method and a computer program for executing the method.

### CROSS-REFERENCE TO RELATED APPLICATIONS

This application is a continuation of International Application No. PCT/EP2014/050243 filed Jan. 8, 2014, which claims the benefit of priority to EP13150790.7, filed Jan. 10, 2013, the contents of which are incorporated by reference herein in their entireties.

### REFERENCE TO SEQUENCE LISTING

This application contains a Sequence Listing submitted as an electronic text file named “31410_WO_SEQUENCE_LSITING_AS_FILED.txt”, having a size in bytes of 1.87 kb, and created on Jun. 10, 2015. The information contained in this electronic file is hereby incorporated by reference in its entirety pursuant to 37 CFR §1.52(e)(5).

### FIELD OF THE INVENTION

The present invention relates to the field of molecular biology and nucleic acid amplification. More specifically it relates to methods and reagents for performing temperature calibration in high resolution melting PCR experiments.

### BACKGROUND OF THE INVENTION

The present description refers to a method and a kit for performing temperature calibration in high resolution melting PCR experiments. The present description further refers to a method for optimal calibration allowing read-out of identical or similar melting temperatures for target and calibrator. The present description further refers to an apparatus for performing the method and a computer program for executing the method.

High Resolution Melting (HRM) is a method to detect unknown nucleic acid variations in target sequences after PCR amplification. Compared to conventional methods like Denaturing Gradient Gel Electrophoresis (DGGE), HRM provides several advantages for mutation scanning. These advantages include lower reagent and sample consumption, less optimization steps and a closed assay format executable in a single real time PCR instrument.

After PCR amplification of target sequences up to a length of approximately 250 base pairs in the presence of a special fluorescent DNA binding dye capable of non-covalently binding to double-stranded nucleic acids (e.g. LightCycler® 480 Resolight Dye, Roche, Cat. No. 04909640001), a HRM step of the generated amplicon is added. As the fluorescent, non-covalent double-stranded DNA binding dye does not inhibit PCR, it can be added to the amplification reaction in saturating concentrations. During the HRM step, the fluorescent, non-covalent double-stranded DNA binding dye is released and differences regarding the amplicon melting profiles between wildtypes, homozygous and heterozygous mutants can be detected (Reed G H, Kent J O, Wittwer C T (2007), Pharmacogenomics 8(6): 597-608; Wittwer C T (2009), Hum. Mutat. 30(6): 857-859; Wittwer et al., U.S. Pat. No. 7,582,429).

Depending on the type of point mutation the observed melting temperature differences can be very small. Single nucleotide polymorphisms (SNPs) typically result in melting temperature shifts between approximately 1.0° C. for SNPs class 1 (C/T and G/A base change) and SNPs class 2 (C/A and G/T base change), approximately 0.5° C. for SNPs class 3 (C/G base change) and approximately 0.2° C. for SNPs class 4 (A/T base change). While heterozygous mutations typically show different fluorescence melting profiles (melting curve shapes) compared to wildtypes, homozygous mutations often result in melting profiles very similar to wildtypes and are only distinguishable by a small temperature shift.

The state of the art regarding HRM exhibits several drawbacks as described herein below. To detect small differences in melting temperature, an extremely high temperature accuracy of the measuring system is required. Real time PCR instruments typically are designed as block-based systems using Peltier elements for precise temperature control. However, the temperature control is subject to physical limitations caused e.g. by calibration of temperature sensors, control of Peltier elements and the geometric fit of individual microwell plates to the mount of the thermal block. These limitations typically result in an observed temperature range of 0.5-1.0° C. between the hottest and the coolest position within the thermal block. Thus, the temperature control within the reaction volume does not allow to distinguish small temperature shifts in HRM experiments with very similar melting profiles characteristic for homozygous mutants compared to their wildtypes.

To correct the heterogeneous temperature distribution in all positions of a block-based thermocycler, two different methods are currently established:

Before HRM experiments are performed on a certain instrument, a separate temperature calibration run is executed using a special calibration plate. The block-specific temperature data for all positions are saved in the instrument's software and are subsequently used in HRM experiments to correct the temperature differences of all positions. This method is established e.g. for Applied Biosystems 7500 and 7900HT real time PCR systems (e.g. MeltDoctor® HRM Calibration Plate, Cat. No. PN4425618) and for Biorad CFX real time PCR systems (e.g. Melt Calibration Kit, Cat. No. 184-5020).

Disadvantages of said calibration method are, when a separate temperature calibration run is performed, experiment-specific causes for temperature inhomogeneity cannot be corrected. These include varying fits of individual microwell plates into the thermal block mount and associated differences in temperature transfer from mount to plate and reaction volume. Furthermore, varying reaction conditions caused e.g. by varying ionic strength (caused by the purification method or the sample material) do influence the observed melting temperature. In addition, heat ageing of the Peltier block cannot be compensated by this method.
1) During the HRM experiment internal temperature calibrators are added to each reaction. The temperature calibrators consist of unlabeled double stranded oligonucleotides that employ melting temperatures below and above the expected melting temperature of the target sequence and are detected using the fluorescent, non-covalent double-stranded DNA binding dye being present in the reaction. Based on the measured well-to-well temperature differences of the calibrators the detected target temperatures are corrected. This method is established e.g. for Idaho Technology's LightScanner® instrument (High Sensitivity Master Mix, Cat. No. HRLS-ASY-0008).

Disadvantages of temperature calibrator method: The detection of the unlabeled internal temperature calibrators is based on release of the same fluorescent, non-covalent double-stranded DNA binding dye used for target mutation detection. Consequently the target melt temperature must not overlap with the calibrator melts. This limits the amplicon size to a range of approximately 40-120 base pairs. Furthermore, depending on the target's G/C-content, the amplicon length has to be optimized to fit into the allowed melting temperature range. In addition, the fluorescence brightness of the amplicon melt must not outperform and consequently hide the internal calibrator signals. Fluorescence brightness strongly depends on the amount of PCR product generated. Therefore, amounts of starting nucleic acid material and concentrations of primers have to be optimized for each target before executing HRM experiments.

The object of the present description is the provision of a method for HRM, which does not show the above mentioned drawbacks.

### SUMMARY OF THE INVENTION

The first aspect of the present description refers to a method for temperature calibration in PCR experiments, wherein the method comprises the following steps of a) providing in each well of a multi-well plate a reaction mixture for amplifying a specific target nucleic acid in a sample comprising a fluorescent, non-covalent double-stranded DNA binding dye, b) providing in each well a double stranded oligonucleotide, wherein a donor chromophore is covalently bound to the first strand of the double stranded oligonucleotide and wherein an acceptor chromophore is covalently bound to the second strand of the double stranded oligonucleotide, c) amplifying in each well the specific target nucleic acid, d) melting in each well the amplified specific target nucleic acid resulting in a decrease of emission of radiation from the fluorescent, non-covalent double-stranded DNA binding dye, and the double stranded oligonucleotide resulting in an increase of emission of radiation from the donor chromophore or a decrease of emission of radiation from the acceptor chromophore by spatially separating donor chromophore and acceptor chromophore, e) monitoring in each well the values of the melting temperature for the amplified specific target nucleic acid by detecting the decrease of emission of radiation from the fluorescent, non-covalent double-stranded DNA binding dye and separately monitoring in each well the values of the melting temperature of the double stranded oligonucleotide by detecting the increase of emission of radiation from the donor chromophore or the decrease of emission of radiation from the acceptor chromophore, f) correcting for each well the melting temperature values for the amplified specific target nucleic acid based on the well-to-well differences of the melting temperature values of the double stranded oligonucleotide.

The second aspect of the present description refers to a kit for performing temperature calibration in PCR experiments as described above, wherein the kit comprises a) all reagents necessary for amplifying a specific target nucleic acid sequence in a sample, b) a fluorescent, non-covalent double-stranded DNA binding dye, c) a double stranded oligonucleotide, wherein a donor chromophore is covalently bound to the first strand of the double stranded oligonucleotide and wherein an acceptor chromophore is covalently bound to the second strand of the double stranded oligonucleotide.

The third aspect of the present description refers to a reaction mixture for performing temperature calibration in PCR experiments as described above, wherein the reaction mixture comprises a) a target nucleic acid sequence, b) all reagents necessary for amplifying the specific target nucleic acid sequence, c) a fluorescent, non-covalent double-stranded DNA binding dye, and d) a double stranded oligonucleotide, wherein a donor chromophore is covalently bound to the first strand of the double stranded oligonucleotide and wherein an acceptor chromophore is covalently bound to the second strand of the double stranded oligonucleotide.

The forth aspect of the present description refers to an apparatus for performing temperature calibration in PCR experiments as described above.

The fifth aspect of the present description refers to a computer program for executing the method for temperature calibration in PCR experiments as described above.

### DESCRIPTION OF THE FIGURES

FIG. 1: The figure shows normalized melting curves of 32 wildtype, 32 heterozygous mutants and 32 homozygous mutants without use of a calibrator as described in example 1. The experiment was performed on an instrument with a thermally uncalibrated PCR block.

FIG. 2: The figure shows normalized melting curves of 32 wildtype, 32 heterozygous mutants and 32 homozygous mutants with use of a calibrator as described in example 1. The experiment was performed on an instrument with a thermally uncalibrated PCR block.

FIG. 3: The figure shows normalized melting curves of six genotype variants without use of a calibrator as described in example 1. The experiment was performed on an instrument with a thermally precalibrated PCR block.

FIG. 4: The figure shows normalized melting curves of six genotype variants with use of a calibrator as described in example 1. The experiment was performed on an instrument with a thermally precalibrated PCR block.

### DETAILED DESCRIPTION OF THE INVENTION

The following definitions are set forth to illustrate and define the meaning and scope of various terms used herein.

The terms “a”, “an” and “the” generally include plural referents, unless the context clearly indicates otherwise.

The term “amplicon” generally refers to selected amplification products which are amplified by a specific set of forward and reverse primers such as those produced from amplification techniques known in the art.

The term “amplification” generally refers to the production of a plurality of nucleic acid molecules from a target nucleic acid wherein primers hybridize to specific sites on the target nucleic acid molecules in order to provide an initiation site for extension by a polymerase. Amplification can be carried out by any method generally known in the art, such as but not limited to: standard PCR, long PCR, hot start PCR, qPCR, RT-PCR and Isothermal Amplification.

The term “calibrator” or “temperature calibrator” is used herein and refers to a double stranded oligonucleotide which carries a FRET pair, the emission wavelength of one counterpart of which can be detected upon melting of the double stranded oligonucleotide. The calibrator is used in high resolution melting experiments in order to determine the temperature differences within the wells of a multi-well plate caused e.g. by irregularities of the thermal block carrying the multi-well plate or by the geometric fit of the individual micro-well plates to the mount of the thermal block. The temperature differences are determined by accurately measuring the melting temperature of the calibrator on the basis of the change regarding the emitted radiation. Said change is a change in intensity (decrease or increase).

The term “complementary” generally refers to the ability to form favorable thermodynamic stability and specific pairing between the bases of two nucleotides at an appropriate temperature and ionic buffer conditions. This pairing is dependent on the hydrogen bonding properties of each nucleotide. The most fundamental examples of this are the hydrogen bond pairs between thymine/adenine and cytosine/guanine bases. In the present description, primers for amplification of target nucleic acids can be both fully complementary over their entire length with a target nucleic acid molecule or “semi-complementary” wherein the primer contains additional, non-complementary sequence minimally capable or incapable of hybridization to the target nucleic acid.

The term “dye” is used to summarize all kinds of light adsorbing molecules and therefore, comprises fluorescent dyes, non-fluorescent dyes and quencher molecules. Quencher molecules are capable of quenching the fluorescence of fluorescent dyes as they are excitable by fluorescent light and dispense energy e.g. by heat. Non-fluorescent dyes are dyes substantially without fluorescence emission in contrast to conventional fluorescent dyes.

The term “fluorescent, non-covalent double-stranded DNA binding dye” refers to a chromophore which is able to bind to double-stranded DNA and enables measurement of DNA formation in qPCR experiments and dissociation in melting analyses. The fluorescent, non-covalent double-stranded DNA binding dye emits radiation in form of light at a certain wavelength when bound to double-stranded DNA. Emission of radiation decreases if the two complementary strands of the double stranded DNA dissociate, e.g. during melting experiments.

The terms “FRET” or “fluorescent resonance energy transfer” or “Foerster resonance energy transfer” can be used interchangeably and refer to a transfer of energy between at least two chromophores, a donor chromophore and an acceptor chromophore. The donor typically transfers the energy to the acceptor when the donor is excited by light radiation with a suitable wavelength. The acceptor typically re-emits the transferred energy in the form of light radiation with a different wavelength. When the acceptor is a “dark” quencher, it dissipates the transferred energy in a form other than light, e.g. in form of heat. Commonly used dark quenchers include BlackHole Quenchers™ (BHQ), (Biosearch Technologies, Inc., Novato, Calif.), Iowa Black™ (Integrated DNA Tech., Inc., Coralville, Iowa), and BlackBerry™ Quencher 650 (BBQ-650) (Berry & Assoc., Dexter, Mich.).

The term “hybridize” generally refers to the base-pairing between different nucleic acid molecules consistent with their nucleotide sequences. The terms “hybridize” and “anneal” can be used interchangeably.

The term “multi-well plate” is used herein as known to the expert skilled in the art and refers to a plate used for analysis of physical, chemical or biological characteristics of one or more samples in parallel. Multi-well plates contain 96, 384, 1536 or 3456 discrete wells. The term also includes other types of reaction devices such as 8-well strips.

The term “mutant” in the context of the present description, means a polynucleotide that comprises one or more base substitutions relative to a corresponding, naturally-occurring or unmodified nucleic acid.

The terms “nucleic acid” or “polynucleotide” can be used interchangeably and refer to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendent moieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. (Science 254:1497-1500, 1991). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The term “oligonucleotide” refers to a nucleic acid that includes at least two nucleic acid monomer units (e.g., nucleotides). An oligonucleotide typically includes from about six to about 175 nucleic acid monomer units, more typically from about eight to about 100 nucleic acid monomer units, and still more typically from about 10 to about 50 nucleic acid monomer units (e.g., about 15, about 20, about 25, about 30, about 35, or more nucleic acid monomer units). The exact size of an oligonucleotide will depend on many factors, including the ultimate function or use of the oligonucleotide. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (Meth. Enzymol. 68:90-99, 1979); the phosphodiester method of Brown et al. (Meth. Enzymol. 68:109-151, 1979); the diethylphosphoramidite method of Beaucage et al. (Tetrahedron Lett. 22:1859-1862, 1981); the triester method of Matteucci et al. (J. Am. Chem. Soc. 103:3185-3191, 1981); automated synthesis methods; or the solid support method of U.S. Pat. No. 4,458,066, or other methods known to those skilled in the art.

The term “primer” generally refers to an oligonucleotide that is able to anneal, or hybridize, to a nucleic acid sequence and allow for extension under sufficient conditions (buffer, dNTPs, polymerase, mono- and divalent salts, temperature, etc.) of the nucleic acid to which the primer is complementary.

The term “qPCR” generally refers to the PCR technique known as real-time quantitative polymerase chain reaction, quantitative polymerase chain reaction or kinetic polymerase chain reaction. This technique simultaneously amplifies and quantifies target nucleic acids using PCR wherein the quantification is by virtue of an intercalating fluorescent dye or sequence-specific probes which contain fluorescent reporter molecules that are only detectable once hybridized to a target nucleic acid.

The term “reaction mixture” is used herein as known to the expert skilled in the art and refers to an aqueous solution comprising various reagents used for amplification of one or more target nucleic acids, including enzymes, aqueous buffers, salts, primers, target nucleic acid, and nucleoside triphosphates. The reaction mixture can be either a complete or incomplete amplification reaction mixture.

A method for temperature calibration in PCR experiments is described herein which overcomes limitations of known temperature calibration methods. During HRM experiments using the method according to the present description a double stranded oligonucleotide is used as a temperature calibrator, which is added to each reaction in a multi-well plate. The double stranded oligonucleotide (herein also referred to as “temperature calibrator” or “calibrator”) carries a FRET pair, the emission wavelength of one counterpart of which can be detected upon melting of the double stranded oligonucleotide. The detected melting temperature values of the double stranded oligonucleotide is subsequently used to correct for each well of a multi-well plate the melting temperature values for an amplified specific target nucleic acid based on the well-to-well differences of the melting temperature values of the double stranded oligonucleotide.

The present description refers to a method for temperature calibration in PCR experiments, wherein the method comprises the steps of a) providing in each well of a multi-well plate a reaction mixture for amplifying a specific target nucleic acid in a sample comprising a fluorescent, non-covalent double-stranded DNA binding dye, b) providing in each well a double stranded oligonucleotide, wherein a donor chromophore is covalently bound to the first strand of the double stranded oligonucleotide and wherein an acceptor chromophore is covalently bound to the second strand of the double stranded oligonucleotide, c) amplifying in each well the specific target nucleic acid, d) melting in each well the amplified specific target nucleic acid resulting in a decrease of emission of radiation from the fluorescent, non-covalent double-stranded DNA binding dye, and the double stranded oligonucleotide resulting in an increase of emission of radiation from the donor chromophore or a decrease of emission of radiation from the acceptor chromophore by spatially separating donor chromophore and acceptor chromophore, e) monitoring in each well the values of the melting temperature for the amplified specific target nucleic acid by detecting the decrease of emission of radiation from the fluorescent, non-covalent double-stranded DNA binding dye and separately monitoring in each well the values of the melting temperature of the double stranded oligonucleotide by detecting the increase of emission of radiation from the donor chromophore or the decrease of emission of radiation from the acceptor chromophore, f) correcting for each well the melting temperature values for the amplified specific target nucleic acid based on the well-to-well differences of the melting temperature values of the double stranded oligonucleotide.

In one embodiment, the specific target nucleic acid comprises a single nucleotide polymorphism (SNP). In another embodiment the specific target nucleic acid comprises more than one SNP. A SNP is a point mutation between corresponding nucleic acid fragments in different samples. Such SNPs change the melting temperature of the nucleic acid fragment by a defined value contained in a sample as compared to the corresponding fragment in another sample (e.g. a reference sample) not exhibiting the same SNP. The differences regarding the melting temperature between corresponding fragments with and without the SNP are in general very small and depend on the type of point mutation. SNPs typically result in melting temperature shifts of 0.2° C. to 1.0° C. between the corresponding fragments. The shifts in melting temperature are i) approximately 1.0° C. for SNPs class 1 (C/T and G/A base change) and SNPs class 2 (C/A and G/T base change), ii) approximately 0.5° C. for SNPs class 3 (C/G base change) and iii) approximately 0.2° C. for SNPs class 4 (A/T base change). Shifts in temperature are used in the present description to determine the presence of a SNP in the specific target nucleic acid as compared to the corresponding target nucleic acid in another sample (e.g. a reference sample).

The donor chromophore is covalently bound at a location within the first strand of the double stranded oligonucleotide and the acceptor chromophore is covalently bound at a location within the second strand of the double stranded oligonucleotide, such that the location within the first strand and the location within the second strand are in close proximity to one another. The donor chromophore is covalently bound at a location within the first strand of the double stranded oligonucleotide and the acceptor chromophore is covalently bound at a location within the second strand of the double stranded oligonucleotide, such that the location within the first strand and the location within the second strand are in an opposite arrangement. The donor chromophore is covalently bound at the 3′-end of the first strand of the double stranded oligonucleotide and the acceptor chromophore is covalently bound at the 5′-end of the second strand of the double stranded oligonucleotide, such that the location within the first strand and the location within the second strand is in an opposite arrangement. The donor chromophore is covalently bound at the 5′-end of the first strand of the double stranded oligonucleotide and the acceptor chromophore is covalently bound at the 3′-end of the second strand of the double stranded oligonucleotide, such that the location within the first strand and the location within the second strand is in an opposite arrangement.

In one embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand are separated from each other by not more than two base pairs.

In another embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand form a complementary base pair.

In a specific embodiment, the donor chromophore is covalently bound to the 5′-end of the first strand of the double stranded oligonucleotide and the acceptor chromophore is covalently bound to the 3′-end of the second strand of the double stranded oligonucleotide or the donor chromophore is covalently bound to the 3′-end of the first strand of the double stranded oligonucleotide and the acceptor chromophore is covalently bound to the 5′-end of the second strand of the double stranded oligonucleotide.

Fluorescent, non-covalent double-stranded DNA binding dyes are well known in the art. Such fluorescent, non-covalent double-stranded DNA binding dyes are for example LC Green®, Idaho Technology; or EvaGreen®, BioRad. In a specific embodiment, the fluorescent, non-covalent double-stranded DNA binding dye is LightCycler® 480 Resolight Dye.

In one embodiment, the donor chromophore is a fluorescent dye, such as VIC, Hex, Yellow555, Red610, Red640, Texas Red, Rox, Cy5 or Cy5.5. In a specific embodiment the donor chromophore is Cy5.

In a specific embodiment, the wavelength of the radiation of the fluorescent, non-covalent double-stranded DNA binding dye and the wavelength of the radiation of the donor chromophore or the acceptor chromophore are separated from each other, enabling detection of both melting events independent of their melting temperature. This has the advantage that the emission wavelength of the fluorescent, non-covalent double-stranded DNA binding dye and the emission wavelength of the donor chromophore or the acceptor chromophore can be distinguished even if the melting temperature of the target nucleic acid and the double stranded oligonucleotide are identical or at least very similar.

In one embodiment, the acceptor chromophore is a quencher molecule, such as BlackHole Quenchers™ (BHQ), (Biosearch Technologies, Inc., Novato, Calif.), Iowa Black™ (Integrated DNA Tech., Inc., Coralville, Iowa), and BlackBerry™ Quencher 650 (BBQ-650) (Berry & Assoc., Dexter, Mich.). In a specific embodiment, the quencher molecule is a dark quencher selected from the group consisting of BHQ-1, BHQ-2, BHQ-3 and BHQ-4. In a more specific embodiment, the quencher molecule is BHQ-3.

In one embodiment, the donor chromophore is a covalently bound fluorescent dye and the acceptor chromophore is a covalently bound quencher molecule. If the fluorescent dye and the quencher are in close proximity to one another in case the two complementary strands of the double stranded oligonucleotide are hybridized to each other, upon irradiation of the fluorescent dye with a certain wavelength, the energy (light) emitted from the fluorescent dye is transferred to the quencher molecule, which converts the energy into heat and no or little emitted radiation of the fluorescent dye can be measured. If the two complementary strands of the double stranded oligonucleotide are separated from each other upon melting, the fluorescent dye and the quencher are separated spatially from one another. In this case, upon irradiation of the fluorescent dye with a certain wavelength, the radiation emitted from the fluorescent dye cannot be transferred to the quencher molecule and an increase of the emitted radiation of the fluorescent dye can be measured. Thus, the melting temperature of the two complementary strands of the double stranded oligonucleotide can be accurately determined by measuring the increase of the emitted radiation of the fluorescent dye.

In another embodiment, the donor chromophore is a first covalently bound fluorescent dye and the acceptor chromophore is a second covalently bound fluorescent dye. If the first covalently bound fluorescent dye and the second covalently bound fluorescent dye are in close proximity to one another in case the two complementary strands of the double stranded oligonucleotide are hybridized to each other, upon irradiation of the first covalently bound fluorescent dye with a certain wavelength, the energy (light) emitted from the first covalently bound fluorescent dye is transferred to the second covalently bound fluorescent dye, which converts the energy and radiation of a certain wavelength is emitted from the second covalently bound fluorescent dye. If the two complementary strands of the double stranded oligonucleotide are melted, the first covalently bound fluorescent dye and the second covalently bound fluorescent dye are separated spatially from one another. In this case, upon irradiation of the first covalently bound fluorescent dye with a certain wavelength, the radiation emitted from the first covalently bound fluorescent dye cannot be transferred to the second covalently bound fluorescent dye any more and an increase of the emitted radiation of the first covalently bound fluorescent dye and a decrease of the emitted radiation of the second covalently bound fluorescent dye can be measured. Thus, the melting temperature of the two complementary strands of the double stranded oligonucleotide can be accurately determined by measuring the increase of the emitted radiation of the first covalently bound fluorescent dye and/or the decrease of the emitted radiation of the second covalently bound fluorescent dye.

In one embodiment, the double stranded oligonucleotide is designed such that at least a part of the values of the melting temperature of the double stranded oligonucleotide are identical to at least a part of the values of the melting temperature of the amplified specific target nucleic acid. In another embodiment, the double stranded oligonucleotide is designed such that the melting temperature of the double stranded oligonucleotide is identical to the melting temperature of the amplified specific target nucleic acid. In another embodiment, the double stranded oligonucleotide is designed such that the melting temperature of the double stranded oligonucleotide differs from the melting temperature of the amplified specific target nucleic acid not more than 10° C. In a specific embodiment, the double stranded oligonucleotide is designed such that the melting temperature of the double stranded oligonucleotide differs from the melting temperature of the amplified specific target nucleic acid not more than 5° C. In another specific embodiment, the double stranded oligonucleotide is designed such that the melting temperature of the double stranded oligonucleotide differs from the melting temperature of the amplified specific target nucleic acid not more than 2° C. By selecting the fluorescent, non-covalent double-stranded DNA binding dye and the donor chromophore such that they emit radiation at different wavelengths the double stranded oligonucleotide can be designed such that the melting temperatures of the target nucleic acid and the double stranded oligonucleotide are identical or at least very similar. Thus in one embodiment, the double stranded oligonucleotide is designed such that the melting temperature of the double stranded oligonucleotide and the melting temperature of the amplified specific target nucleic acid is identical.

Excitation and emission wavelengths of the donor chromophore (such as Cy5) of the double stranded oligonucleotide are different from the excitation and emission wavelengths of the fluorescent, non-covalent double-stranded DNA binding dye (such as LightCycler® 480 Resolight Dye). Melting of the double stranded oligonucleotide can be detected at a wavelength range separate from the detection wavelength of the fluorescent, non covalent double-stranded DNA binding dye. Consequently, the melting temperature of the calibrator may overlap with the melting temperature of the target. This allows designing both melting temperatures close together and thus enable calibration at exactly the relevant temperature. As position-to-position temperature differences in multi-well plates are not constant over the temperature range applied in HRM, it is of special advantage to provide a calibration method enabling measurement of identical melting temperatures of the double stranded oligonucleotide and the target nucleic acid. Moreover, as the melting temperature of the target nucleic acid and the fluorescence intensity do not influence the signal detected from the double stranded oligonucleotide, there is no need for optimizing the amount of target nucleic acid or the concentration of the primers to generate limited product amounts that do not hide the signal of the double stranded oligonucleotide.

The double stranded oligonucleotide consists of two complementary strands, the first strand of the double stranded oligonucleotide and the second strand of the double stranded oligonucleotide. In one embodiment, the first strand and the second strand each comprises 10 to 40 nucleotides. In a specific embodiment, the first strand and the second strand each comprises 20 to 30 nucleotides. In an even more specific embodiment, the first strand and the second strand each comprises 25 nucleotides.

In one embodiment, the 5′-end of the first strand is covalently bound to a donor chromophore, such as Cy 5, and the 3′-end of the first strand is phosphorylated. The 3′-end of the second strand is covalently bound to a dark quencher, such as BHQ-3. In another embodiment, the 5′-end of the second strand is covalently bound to a donor chromophore, such as Cy 5, and the 3′-end of the second strand is phosphorylated. The 3′-end of the first strand is covalently bound to a dark quencher, such as BHQ-3.

In a specific embodiment, the first strand (SEQ ID NO:01) and the complementary second strand (SEQ ID NO:02) comprise the following sequences and labels:

SEQ ID NO: 01
5′-Cy5-TGG GGG TGG GGG TGG GGG TGG GGG T-P-3′
SEQ ID NO: 02
5′-ACC CCC ACC CCC ACC CCC ACC CCC A-BHQ-3-3′

As already mentioned above, it is advantageous to design the double stranded oligonucleotide (calibrator) such that at least a part of the values of the melting temperature of the double stranded oligonucleotide are identical to at least a part of the values of the melting temperature of the amplified specific target nucleic acid. Therefore, the calibrator can comprise any sequence dependent on the target nucleic acids amplified and analyzed. SEQ ID NO:01 and SEQ ID NO:02 has to be regarded as one single possibility, which was found to be suitable as a calibrator in the present examples 1 to 3.

In a specific embodiment, the method for temperature calibration in PCR experiments comprises the steps of a) providing in each well of a multi-well plate a reaction mixture for amplifying a specific target nucleic acid in a sample, wherein the specific target nucleic acid comprises a single nucleotide polymorphism, and LightCycler® 480 Resolight Dye, b) providing in each well a double stranded oligonucleotide, wherein the fluorescent dye Cy5 is covalently bound to the first strand of the double stranded oligonucleotide and wherein the dark quencher BHQ-3 is covalently bound to the second strand of the double stranded oligonucleotide, wherein the fluorescent dye Cy5 is covalently bound to a nucleotide within the first strand of the double stranded oligonucleotide and the dark quencher BHQ-3 is covalently bound to a nucleotide within the second strand of the double stranded oligonucleotide, wherein the nucleotide within the first strand and the nucleotide within the second strand form a complementary base pair, c) amplifying in each well the specific target nucleic acid, d) melting in each well the amplified specific target nucleic acid resulting in a decrease of emission of radiation from LightCycler® 480 Resolight Dye, and the double stranded oligonucleotide resulting in an increase of emission of radiation from Cy5 by spatially separating the fluorescent dye Cy5 and the dark quencher BHQ-3, e) monitoring in each well the values of the melting temperature for the amplified specific target nucleic acid by detecting the decrease of emission of radiation from LightCycler® 480 Resolight Dye and separately monitoring in each well the values of the melting temperature of the double stranded oligonucleotide by detecting the increase of emission of radiation from the fluorescent dye Cy5, f) correcting for each well the melting temperature values for the amplified specific target nucleic acid based on the well-to-well differences of the melting temperature values of the double stranded oligonucleotide.

The present description further refers to a kit for performing temperature calibration in PCR experiments as described above, wherein the kit comprises a) all reagents necessary for amplifying a specific target nucleic acid sequence in a sample, b) a fluorescent, non-covalent double-stranded DNA binding dye, c) a double stranded oligonucleotide, wherein a donor chromophore is covalently bound to the first strand of the double stranded oligonucleotide and wherein an acceptor chromophore is covalently bound to the second strand of the double stranded oligonucleotide.

In one embodiment, the specific target nucleic acid comprises a single nucleotide polymorphism.

In one embodiment, the donor chromophore is covalently bound at a location within the first strand of the double stranded oligonucleotide and the acceptor chromophore is covalently bound at a location within the second strand of the double stranded oligonucleotide, such that the location within the first strand and the location within the second strand is in close proximity to one another. In a specific embodiment, the location within the first strand and the location within the second strand are in opposed positions of the double stranded oligonucleotide.

In a specific embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand are separated from each other by not more than two base pairs.

In another embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand form a complementary base pair.

In one embodiment, the emission wavelength of the fluorescent, non-covalent double-stranded DNA binding dye and the emission wavelength of the donor chromophore are separated from each other.

In a specific embodiment, the fluorescent, non-covalent double-stranded DNA binding dye is LightCycler® 480 Resolight Dye. In one embodiment, the donor chromophore is Cy5. In one embodiment, the acceptor dye is a quencher molecule. In a specific embodiment, the quencher molecule is a dark quencher selected from the group consisting of BHQ-1, BHQ-2, BHQ-3 and BHQ-4. In a more specific embodiment, the quencher molecule is BHQ-3.

The present description further refers to a reaction mixture for performing temperature calibration in PCR experiments as described above, wherein the reaction mixture comprises a) a target nucleic acid sequence, b) all reagents necessary for amplifying the specific target nucleic acid sequence, c) a fluorescent, non-covalent double-stranded DNA binding dye, and d) a double stranded oligonucleotide, wherein a donor chromophore is covalently bound to the first strand of the double stranded oligonucleotide and wherein an acceptor chromophore is covalently bound to the second strand of the double stranded oligonucleotide.

In one embodiment, the target nucleic acid comprises a single nucleotide polymorphism.

In one embodiment, the reagents necessary for amplifying the target nucleic acid sequence comprises a buffer, dNTPs, polymerase, mono- and divalent salts, a forward primer and a reverse primer.

In one embodiment, the donor chromophore is covalently bound at a location within the first strand of the double stranded oligonucleotide and the acceptor chromophore is covalently bound at a location within the second strand of the double stranded oligonucleotide, such that the location within the first strand and the location within the second strand is in close proximity to one another. In a specific embodiment, the location within the first strand and the location within the second strand are in opposed positions of the double stranded oligonucleotide.

In a specific embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand are separated from each other by not more than two base pairs.

In another embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand form a complementary base pair.

In one embodiment, the emission wavelength of the fluorescent, non-covalent double-stranded DNA binding dye and the emission wavelength of the donor chromophore are separated from each other.

In a specific embodiment, the fluorescent, non-covalent double-stranded DNA binding dye is LightCycler® 480 Resolight Dye. In one embodiment, the donor chromophore is Cy5. In one embodiment, the acceptor dye is a quencher molecule. In a specific embodiment, the quencher molecule is a dark quencher selected from the group consisting of BHQ-1, BHQ-2, BHQ-3 and BHQ-4. In a more specific embodiment, the quencher molecule is BHQ-3.

The present description further refers to an apparatus for performing temperature calibration in PCR experiments as described above. Thus, the present description refers to an apparatus for performing a method for temperature calibration in PCR experiments, wherein the method comprises the steps of a) providing in each well of a multi-well plate a reaction mixture for amplifying a specific target nucleic acid in a sample comprising a fluorescent, non-covalent double-stranded DNA binding dye, b) providing in each well a double stranded oligonucleotide, wherein a donor chromophore is covalently bound to the first strand of the double stranded oligonucleotide and wherein an acceptor chromophore is covalently bound to the second strand of the double stranded oligonucleotide, c) amplifying in each well the specific target nucleic acid, d) melting in each well the amplified specific target nucleic acid resulting in a decrease of emission of radiation from the fluorescent, non-covalent double-stranded DNA binding dye, and the double stranded oligonucleotide resulting in an increase of emission of radiation from the donor chromophore or a decrease of emission of radiation from the acceptor chromophore by spatially separating donor chromophore and acceptor chromophore, e) monitoring in each well the values of the melting temperature for the amplified specific target nucleic acid by detecting the decrease of emission of radiation from the fluorescent, non-covalent double-stranded DNA binding dye and separately monitoring in each well the values of the melting temperature of the double stranded oligonucleotide by detecting the increase of emission of radiation from the donor chromophore or the decrease of emission of radiation from the acceptor chromophore, f) correcting for each well the melting temperature values for the amplified specific target nucleic acid based on the well-to-well differences of the melting temperature values of the double stranded oligonucleotide.

In one embodiment, the target nucleic acid comprises a single nucleotide polymorphism.

In one embodiment, the donor chromophore is covalently bound at a location within the first strand of the double stranded oligonucleotide and the acceptor chromophore is covalently bound at a location within the second strand of the double stranded oligonucleotide, such that the location within the first strand and the location within the second strand is in close proximity to one another. In a specific embodiment, the location within the first strand and the location within the second strand are in opposed positions of the double stranded oligonucleotide.

In a specific embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand are separated from each other by not more than two base pairs.

In another embodiment, the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand form a complementary base pair.

In one embodiment, the emission wavelength of the fluorescent, non-covalent double-stranded DNA binding dye and the emission wavelength of the donor chromophore are separated from each other.

In a specific embodiment, the fluorescent, non-covalent double-stranded DNA binding dye is LightCycler® 480 Resolight Dye. In one embodiment, the donor chromophore is Cy5. In one embodiment, the acceptor dye is a quencher molecule. In a specific embodiment, the quencher molecule is a dark quencher selected from the group consisting of BHQ-1, BHQ-2, BHQ-3 and BHQ-4. In a more specific embodiment, the quencher molecule is BHQ-3.

The present description further refers to a computer program for executing a method as described above. Thus, the present description refers to a computer program for executing a method for temperature calibration in PCR experiments, wherein the method comprises the steps of a) providing in each well of a multi-well plate a reaction mixture for amplifying a specific target nucleic acid in a sample comprising a fluorescent, non-covalent double-stranded DNA binding dye, b) providing in each well a double stranded oligonucleotide, wherein a donor chromophore is covalently bound to the first strand of the double stranded oligonucleotide and wherein an acceptor chromophore is covalently bound to the second strand of the double stranded oligonucleotide, c) amplifying in each well the specific target nucleic acid, d) melting in each well the amplified specific target nucleic acid resulting in a decrease of emission of radiation from the fluorescent, non-covalent double-stranded DNA binding dye, and the double stranded oligonucleotide resulting in an increase of emission of radiation from the donor chromophore or a decrease of emission of radiation from the acceptor chromophore by spatially separating donor chromophore and acceptor chromophore, e) monitoring in each well the values of the melting temperature for the amplified specific target nucleic acid by detecting the decrease of emission of radiation from the fluorescent, non-covalent double-stranded DNA binding dye and separately monitoring in each well the values of the melting temperature of the double stranded oligonucleotide by detecting the increase of emission of radiation from the donor chromophore or the decrease of emission of radiation from the acceptor chromophore, f) correcting for each well the melting temperature values for the amplified specific target nucleic acid based on the well-to-well differences of the melting temperature values of the double stranded oligonucleotide. In one embodiment, the specific target nucleic acid comprises a single nucleotide polymorphism.

The optimal design, sequence and labeling of the double stranded oligonucleotide (calibrator) has to be determined in order to achieve the highest possible benefit from the invention described herein. In particular, the following features of the calibrator are advantageous compared to the state of the art:
a) Tm of the calibrator should be comparable to Tm of a typical target nucleic acid, as position-to-position temperature differences depend on the target temperature being analyzed.
b) No inhibition of target amplification efficiency. This is important in order to get objective results from the PCR analysis which is completely unaffected from the presence of the calibrator.
c) Minimized overlap of the emission wavelength of the fluorescent, non-covalent DNA binding dye and the fluorescent dye of the calibrator to reduce interference of melting curve shapes of target nucleic acid and calibrator.
d) Generate sufficient melting signal intensity to provide a reliable read-out of the calibrator Tm.

### EXAMPLES

The following examples 1 to 3 are provided to aid the understanding of the present description, the true scope of which is set forth in the appended claims. It is understood that modifications can be made in the procedures set forth without departing from the spirit of the invention.

### Example 1

### Design of the Calibrator

The calibrator consists of two 25 mer complementary strands. One strand is labeled at the 5′-end with the fluorescent dye Cy 5 and is phosphorylated at the 3′-end. The other strand is labeled at 3′-end with the dark quencher BHQ-3 (Biosearch Technologies).

SEQ ID NO: 01
5′-Cy5-TGG GGG TGG GGG TGG GGG TGG GGG T-P-3′
SEQ ID NO: 02
5′-ACC CCC ACC CCC ACC CCC ACC CCC A-BHQ-3-3′

The experiments provided below are each performed without and with the use of a calibrator according to the present description, respectively.

### Example 2

### Improved Temperature Resolution by Use of a Calibrator on a Thermally Uncalibrated PCR Block

Two Single Nucleotide Polymorphism (SNP) regions were amplified from 2 ng human genomic DNA (purified from different human blood samples).

An ADD1 gene region was amplified using the following primer sequences:

SEQ ID NO: 03
5′-GAT GGC TGA ACT CTG GC-3′
SEQ ID NO: 04
5′-CGA CTT GGG ACT GCT TC-3′

A Cyp2C9 gene region was amplified using the following primer sequences:

SEQ ID NO: 05
5′-CGT TTC TCC CTC ATG ACG-3′
SEQ ID NO: 06
5′-TCA GTG ATA TGG AGT AGG GTC-3′

The following PCR and melting protocol was applied using a LightCycler® 96 real time PCR instrument (prototype instrument from Roche).

| | | | | | Ramp | | | |
|---|---|---|---|---|---|---|---|---|
| | | T | | | Rate | Acquisitions | Acquisition | Analysis |
| Program | Cycles | (° C.) | Hold | | (° C./s) | (per ° C.) | Mode | Mode |
| Preincubation | 1 | 95 | 10 | min | 4.4 | | | |
| Amplification | 45 | 95 | 10 | sec | 4.4 | | None | Quantifi- |
| | | 60 | 15 | sec | 2.2 | | None | cation |
| | | 72 | 15 | sec | 4.4 | | Single | |
| HRM | 1 | 95 | 1 | min | 4.4 | | None | Melting |
| | | 40 | 1 | min | 2.2 | | None | Curves |
| | | 65 | 1 | sec | 1 | | None | |
| | | 95 | | | | 15 | Contin- | |
| | | | | | | | uous | |
| Cooling | 1 | 40 | 30 | sec | 2.2 | | None | None |

Observed Results:

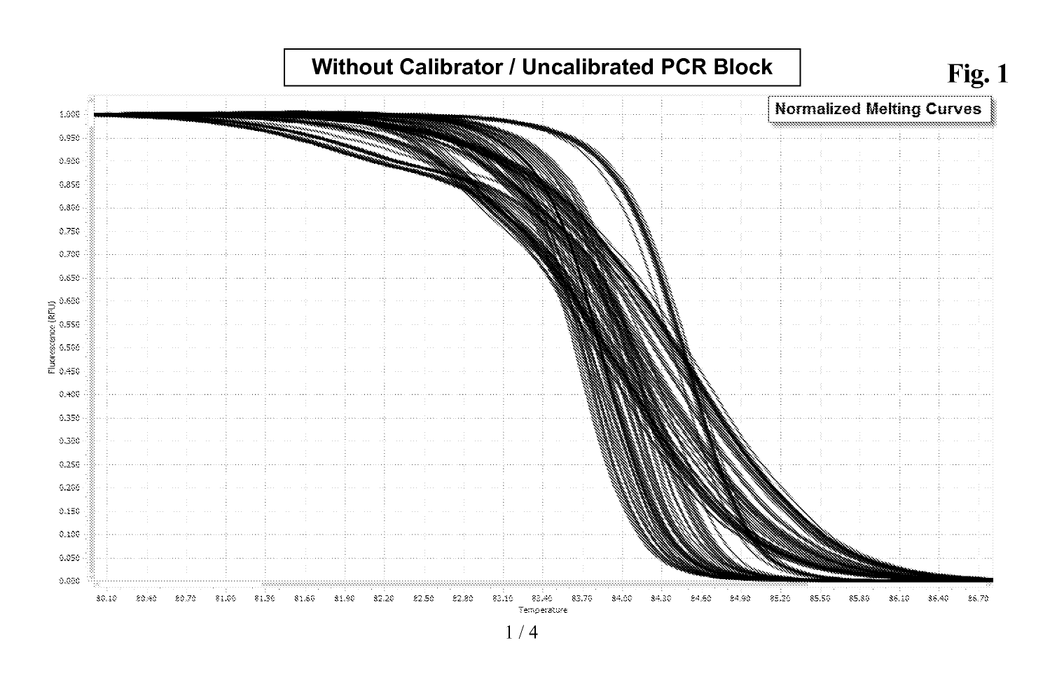

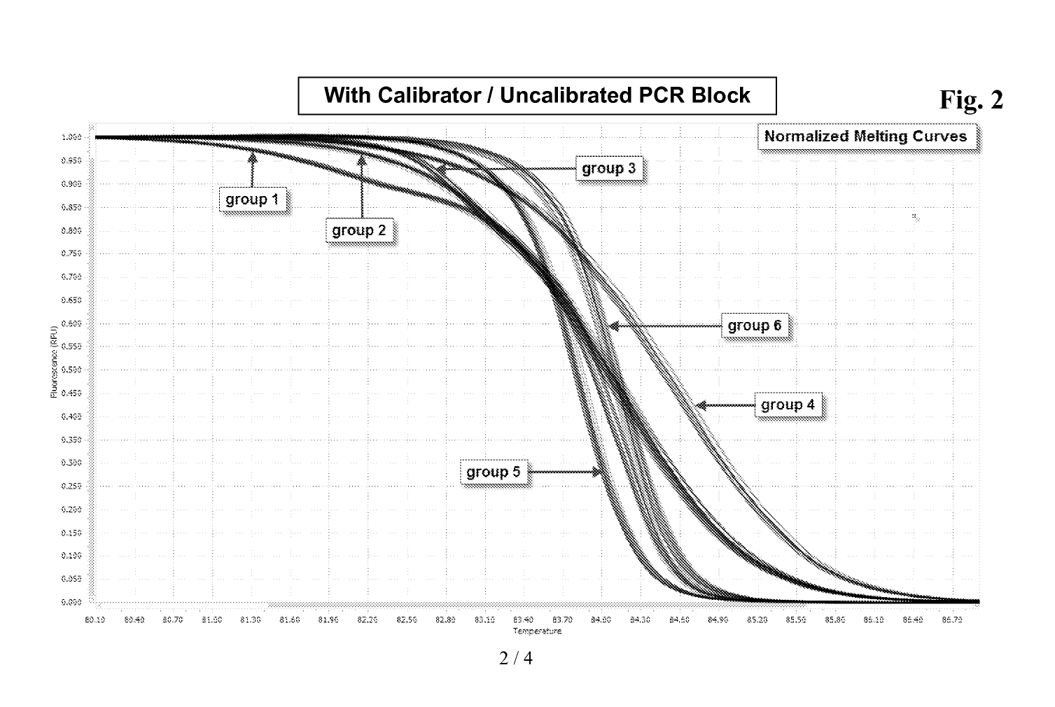

As can be clearly taken from FIG. 1, six different genotypes cannot be distinguished without using the calibrator according to the present description on an uncalibrated PCR-block. However, if the calibrator according to the present description is introduced into the experiment, a clear differentiation of the six groups is possible on an uncalibrated PCR-block (FIG. 2).

### Example 3

### Improved Temperature Resolution by Use of a Calibrator on a Thermally Precalibrated PCR Block

One Single Nucleotide Polymorphism (SNP) region was amplified from 88 different human genomic DNAs (purified from different human blood samples).

A TNF alpha gene region was amplified using the following primer sequences:

SEQ ID NO: 07
5′-GGG CTA TGG AAG TCG AGT A-3′
SEQ ID NO: 08
5′-CGT CCC CTG TAT CCA TAC C-3′

The following PCR and melting protocol was applied using a LightCycler™ 96 real time PCR instrument (prototype instrument from Roche Applied Science).

| | | | | | Ramp | | | |
|---|---|---|---|---|---|---|---|---|
| | | T | | | Rate | Acquisitions | Acquisition | Analysis |
| Program | Cycles | (° C.) | Hold | | (° C./s) | (per ° C.) | Mode | Mode |
| Preincubation | 1 | 95 | 10 | min | 4.4 | | | |
| Amplification | 45 | 95 | 10 | sec | 4.4 | | None | Quantifi- |
| | | 60 | 15 | sec | 2.2 | | None | cation |
| | | 72 | 15 | sec | 4.4 | | Single | |
| HRM | 1 | 95 | 1 | min | 4.4 | | None | Melting |
| | | 40 | 1 | min | 2.2 | | None | Curves |
| | | 65 | 1 | sec | 1 | | None | |
| | | 95 | | | | 15 | Contin- | |
| | | | | | | | uous | |
| Cooling | 1 | 40 | 30 | sec | 2.2 | | None | None |

Observed Results:

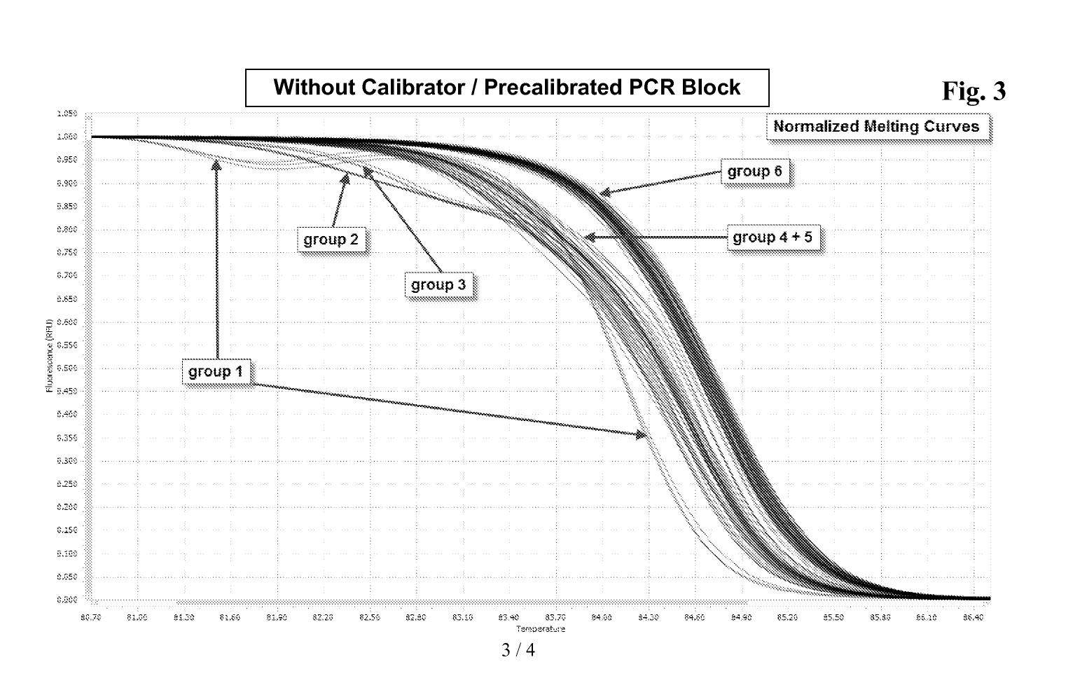

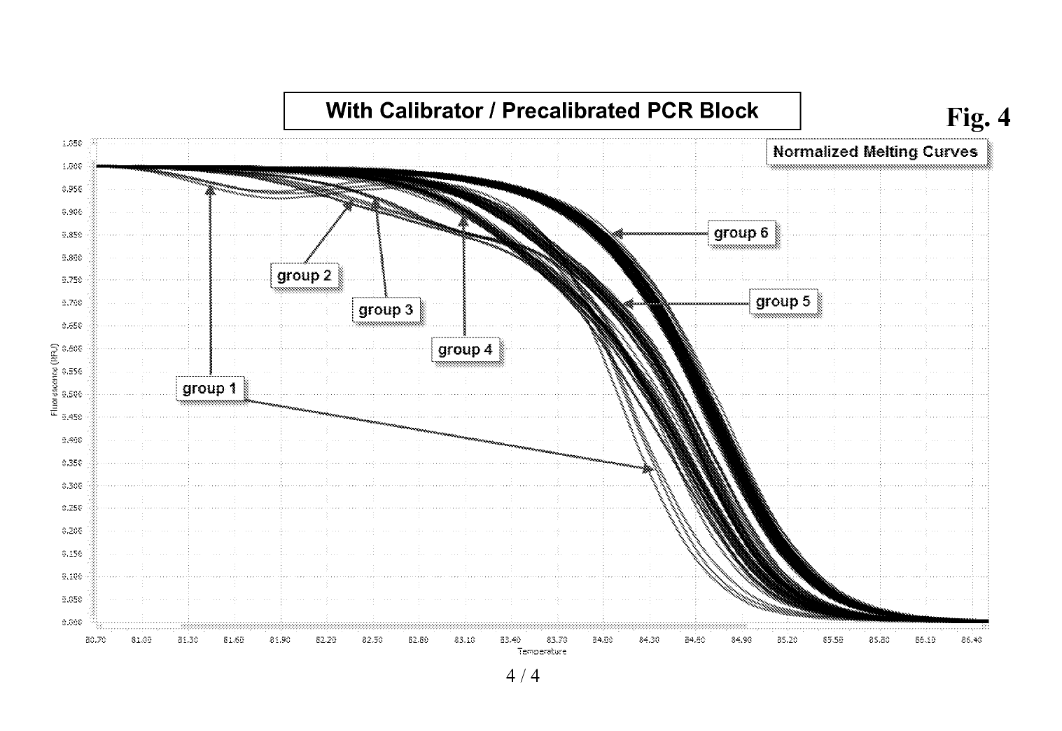

As can be clearly taken from FIG. 3, six different genotypes cannot clearly be distinguished without using the calibrator according to the present description on a precalibrated PCR-block. However, if the calibrator according to the present description is introduced into the experiment, a clear differentiation of the six groups is possible on a precalibrated PCR-block (FIG. 4). The Experiment shows that the effect of the calibrator according to the present description improves the differentiation between different genotypes even if experiments are performed on an instrument with a precalibrated PCR-block.

1. A method for temperature calibration in PCR experiments, wherein the method comprises the following steps:
a. Providing in each well of a multi-well plate a reaction mixture for amplifying a specific target nucleic acid in a sample comprising a fluorescent, non-covalent double-stranded DNA binding dye,
b. Providing in each well a double stranded oligonucleotide, wherein a donor chromophore is covalently bound to the first strand of the double stranded oligonucleotide and wherein an acceptor chromophore is covalently bound to the second strand of the double stranded oligonucleotide,
c. Amplifying in each well the specific target nucleic acid,
d. Melting in each well the amplified specific target nucleic acid resulting in a decrease of emission of radiation from the fluorescent, non-covalent double-stranded DNA binding dye, and the double stranded oligonucleotide resulting in an increase of emission of radiation from the donor chromophore or a decrease of emission of radiation from the acceptor chromophore by spatially separating donor chromophore and acceptor chromophore,
e. Monitoring in each well the values of the melting temperature for the amplified specific target nucleic acid by detecting the decrease of emission of radiation from the fluorescent, non-covalent double-stranded DNA binding dye and separately monitoring in each well the values of the melting temperature of the double stranded oligonucleotide by detecting the increase of emission of radiation from the donor chromophore or the decrease of emission of radiation from the acceptor chromophore,
f. Correcting for each well the melting temperature values for the amplified specific target nucleic acid based on the well-to-well differences of the melting temperature values of the double stranded oligonucleotide.

2. The method of claim 1, wherein the specific target nucleic acid comprises a single nucleotide polymorphism.

3. The method of claim 1, wherein the donor chromophore is covalently bound to a nucleotide within the first strand of the double stranded oligonucleotide and the acceptor chromophore is covalently bound to a nucleotide within the second strand of the double stranded oligonucleotide, wherein the nucleotide within the first strand and the nucleotide within the second strand form a complementary base pair.

4. The method of claim 3, wherein the donor chromophore is covalently bound to the 5′-end of the first strand of the double stranded oligonucleotide and the acceptor chromophore is covalently bound to the 3′-end of the second strand of the double stranded oligonucleotide or wherein the donor chromophore is covalently bound to the 3′-end of the first strand of the double stranded oligonucleotide and the acceptor chromophore is covalently bound to the 5′-end of the second strand of the double stranded oligonucleotide.

5. The method of claim 1, wherein the wavelength of the radiation of the fluorescent, non-covalent double-stranded DNA binding dye and the wavelength of the radiation of the donor chromophore are separated from each other.

6. The method of claim 1, wherein the fluorescent, non-covalent double-stranded DNA binding dye is LightCycler® 480 Resolight Dye.

7. The method of claim 1, wherein the donor chromophore is Cy5.

8. The method of claim 1, wherein the acceptor chromophore is a quencher molecule.

9. The method of claim 8, wherein the quencher molecule is a dark quencher selected from the group consisting of BHQ-1, BHQ-2, BHQ-3 and BHQ-4.

10. The method of claim 1, wherein the double stranded oligonucleotide is designed such that the melting temperature of the double stranded oligonucleotide differs from the melting temperature of the amplified specific target nucleic acid not more than 5° C.

11. The method of claim 1, wherein the double stranded oligonucleotide is designed such that the melting temperature of the double stranded oligonucleotide and the melting temperature of the amplified specific target nucleic acid is identical.

12. A kit for performing a method for temperature calibration in PCR experiments according to claim 1, wherein the kit comprises:
a. All reagents necessary for amplifying a specific target nucleic acid sequence in a sample,
b. A fluorescent, non-covalent double-stranded DNA binding dye,
c. A double stranded oligonucleotide, wherein a donor chromophore is covalently bound to the first strand of the double stranded oligonucleotide and wherein an acceptor chromophore is covalently bound to the second strand of the double stranded oligonucleotide.

13. A reaction mixture for performing a method for temperature calibration in PCR experiments according to claim 1, wherein the reaction mixture comprises:
a. A target nucleic acid sequence,
b. All reagents necessary for amplifying the specific target nucleic acid sequence,
c. A fluorescent, non-covalent double-stranded DNA binding dye,
d. A double stranded oligonucleotide, wherein a donor chromophore is covalently bound to the first strand of the double stranded oligonucleotide and wherein an acceptor chromophore is covalently bound to the second strand of the double stranded oligonucleotide.


SEQUENCE LISTING (8 sequences)

SEQ ID NO: 1 - DNA, 25 bp, Artificial Sequence
  Oligonucleotide-first strand
tgggggtgggggtgggggtgggggt

SEQ ID NO: 2 - DNA, 25 bp, Artificial Sequence
  Oligonucleotide-second complementary strand
acccccacccccacccccaccccca

SEQ ID NO: 3 - DNA, 17 bp, Homo sapiens
gatggctgaactctggc

SEQ ID NO: 4 - DNA, 17 bp, Homo sapiens
cgacttgggactgcttc

SEQ ID NO: 5 - DNA, 18 bp, Homo sapiens
cgtttctccctcatgacg

SEQ ID NO: 6 - DNA, 21 bp, Homo sapiens
tcagtgatatggagtagggtc

SEQ ID NO: 7 - DNA, 19 bp, Homo sapiens
gggctatggaagtcgagta

SEQ ID NO: 8 - DNA, 19 bp, Homo sapiens
cgtcccctgtatccatacc